(19) United States
(12) Patent Application Publication
(10) Pub. No.: US 2003/0170209 A1
(43) Pub. Date: Sep. 11, 2003

(54) Use of a vector comprising a nucleic acid coding for an anti-angiogenic factor for treating corneal neovascularization

(76) Inventor: Marc Abitbol, Paris (FR)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/169,180

(22) PCT Filed: Dec. 21, 2000
(86) PCT No.: PCT/FR00/03653

(30) Foreign Application Priority Data
Dec. 30, 1999 (FR) 99/167780

(51) Int. Cl. A61K 48/00; C12N 15/861

(52) U.S. Cl. 424/93.2; 514/44; 435/455; 435/456

(57) ABSTRACT

The invention concerns the use of a vector comprising a nucleic acid coding for an anti-angiogenic factor for preventing, improving and/or treating corneal neovascularization.

The present invention relates to the use of a vector comprising a nucleic acid encoding an anti-angiogenic factor for the prevention, improvement and/or treatment of neovascularizations of the cornea. It also relates to pharmaceutical compositions and to a device enabling local and efficient administration of this vector.

Keratopathies are pathologies of the cornea of traumatic, chemical, infectious or genetic origin. The ones most frequently encountered, such as herpes or zoster keratopathies (neuroparalytic, keratic, ophthalmic zosta), originate from viral infections. Traumatic keratopathies are caused by projections of small objects such as fragments of a windscreen during road traffic accidents, and chemical keratopathies can be due to the projection of chemical substances on to the eyeball.

This set of pathologies is often complicated by neovascularization which is largely responsible for opacification of the cornea leading to blindness. The only treatment which is currently available for restoring the sight of a patient suffering from blinding keratopathy complicated by neovascularization is corneal transplantation. Although efficient, this approach is, however, considerably limited by the lack of grafts: thus, in France, an annual deficit of 3000 grafts is observed. This point illustrates the need for new treatments for keratopathies which cause blindness.

The eye consists of an anterior segment which comprises the cornea, the anterior chamber filled with aqueous humor, the iris and the lens, and a posterior segment of which the retina, the choroid membrane and the posterior portion of the sclera form part. The cornea comprises, from the surface towards the back of the eye, the corneal epithelium, the Bowman's basal membrane, the corneal stroma and the Descemet's membrane on which the corneal endothelium rests. The cornea, which is a fibrous and transparent envelope, contains no blood vessels or lymphatic vessels. It is nourished by diffusion of metabolites from the aqueous humor and from the blood vessels of the limbus (sclerocorneal junction), and receives part of the O₂ directly from the outside environment.

The blood network of the eye derives from the ophthalmic artery through two different systems; the retinal vessels and the uveal vascular system which comprises the vascular networks of the iris, the ciliary body and the choroid membrane. This vascular tree may be modified in pathological circumstances which are characterized by a decrease in blood flow or in the amount of oxygen. Neovessels are then borne of small vascular loops derived from venules, at the edge of the ischemic zones. The basal membrane of these venules breaks up under the effect of proteolytic enzymes and thus allows migration of endothelial cells. These endothelial cells change in morphology, multiply and then grow hollow with vacuoles which will progressively become confluent and organized to form true vascular lumina. The leakiness of the interendothelial junctions, and the interruption of the basal membrane of these neovessels increases their permeability and leads to extravasation of erythrocytes.

This neovascularization, or angiogenesis, is thus at the origin of most diseases of the vision which bring about a decline in visual acuity, or even blindness. Proliferation of the limbic vessels in the superficial layers of the cornea appears in many pathological states such as certain viral keratoconjunctiviteses, in particular those caused by the herpes virus, and edema. Another form of neovascularizatibn which may lead to blindness is encountered in cases of pterygia in which the membrane of conjunctival origin with vessel-carrying structures progresses from the peribulbar conjunctiva to the corneal surface, or even deeper, to the corneal stroma. Pterygia may also make it necessary to perform corneal transplants, which are sometimes very difficult and may, once again, cause corneal neovascularization.

The inventors have developed a particularly efficient method for preventing and/or treating corneal neovascularization consisting of administration to the eye of a pharmaceutical composition comprising a vector encoding an anti-angiogenic factor, combined with application of a soft contact lens on to the cornea. The contact lens can be applied prior to, concomitantly with, or after administration of the composition comprising the vector. Preferably, the administration of the composition containing the vector is concomitant with the application of the contact lens, and more preferably the administration takes place by prior impregnation of the contact lens with the composition comprising said vector before the application of said lens on to the cornea.

The pharmaceutical composition comprising the vector can be in any form which is adapted for use on the eyes and in particular in the form of an eyewash or an ophthalmic ointment. Preferably, the pharmaceutical composition is in the form of an eyewash.

To study the effect of anti-angiogenic factors in the prevention and treatment of ocular pathologies linked to neovascularization, the applicant has used two animal models of rats which have corneal neovascularization. Transfer of nucleic acids encoding anti-angiogenic factors was carried out by means of defective recombinant adenoviruses encoding various anti-angiogenic factors administered by ocular instillation or by application of soft lenses which were presoaked in a solution containing adenoviruses. Unexpectedly, the applicant demonstrated that the transfer of a vector comprising a nucleic acid encoding an anti-angiogenic factor, at the level of the cornea, by means of a contact lens which has been presoaked in a solution comprising said vector makes it possible to prevent and to treat corneal neovascularization with an efficacy so far unequalled.

Among the various anti-angiogenic factors which can be used in the context of the present invention, mention may be made of in particular: the N-terminal fragment of the plasminogen activator uPA (ATF) (Appella et al. J. Biol. Chem. 262, 4437-4440 (1987)), angiostatin (M. O'Reilly et al. Cell 79, 1157-1164 (1994)) and in particular angiostatin K3 (1-333 N-terminal fragment of human plasminogen), endostatin (M. O'Reilly et al. Cell 88, 277-285 (1997)), the 16 kDa fragment of prolactin (C. Clapp et al. Endocrinol. 133, 1292-1299 (1993)) or platelet factor 4 PF-4 (S. K. Gupta et al. P.N.A.S. USA 92, 7799-7803 (1995)).

A first subject of the invention relates to the use of a vector comprising a nucleic acid encoding at least one anti-angiogenic factor for the preparation of a pharmaceutical composition intended to be administered by impregnation of a soft lens and application of said lens on to the cornea for the prevention, improvement and/or treatment of corneal neovascularization.

Preferably, the nucleic acid encoding the anti-angiogenic factor is a nucleic acid encoding a polypeptide chosen from the N-terminal fragment of the plasminogen activator uPA (ATF), angiostatin, angiostatin K3, endostatin, the 16 kDa fragment of prolactin or platelet factor 4 (PF-4) or a combination of nucleic acids encoding at least two of these factors. More preferably, the anti-angiogenic factor is chosen from the N-terminal fragment of the plasminogen activator uPA (ATF) and angiostatin K3.

According to one particular embodiment, the anti-angiogenic factor is a variant of the abovementioned factors. For the purposes of the present invention, “variant” of a polypeptide or of a protein is taken to mean any analog, fragment, derivative or mutated form which is derived from a polypeptide or from a protein and which retains the anti-angiogenic function of said polypeptide or of said protein. Different variants of a polypeptide or of a protein can exist naturally. These variants can be allelic variations characterized by differences in the nucleotide sequence of structural genes encoding the protein or can result from differential splicing or from post-translational modifications. These variants can be obtained by substitution, deletion, addition and/or modification of one or more amino acid residues. These modifications can be carried out by any technique known to persons skilled in the art.

These variants are in particular molecules which have a greater affinity for their binding sites, sequences which allow improved expression in vivo, molecules which have greater resistance to proteases, and molecules which have greater therapeutic efficacy or fewer side effects, or possibly which have novel biological properties.

Other variants which can be used in the context of the invention are in particular molecules in which one or more residues have been substituted, derivatives obtained by deletion of regions which are relatively uninvolved or not at all involved in the interaction with the binding sites under consideration, or which express an unwanted activity, and derivatives which comprise additional residues relative to the native sequence, such as for example a secretion signal and/or a junction peptide.

The DNA sequence encoding the anti-angiogenic factor used in the context of the present invention can be a cDNA, a genomic DNA (gDNA) or a hybrid construction consisting of, for example, a cDNA into which one or more introns would be inserted. It can be a nucleic acid of animal or human origin; preferably it is a nucleic acid of human origin. It can also be synthetic or semisynthetic sequences. Particularly advantageously, a cDNA or a gDNA is used. In particular, the use of a gDNA can enable better expression in human cells.

Advantageously, the sequence encoding the anti-angiogenic factor is placed under the control of signals which allow its expression in the cells of the corneal epithelium. Preferably, they are heterologous expression signals, i.e. signals other than those naturally responsible for the expression of the anti-angiogenic factor. They can be, in particular, sequences which are responsible for the expression of other proteins, or synthetic sequences. In particular, they can be promoter sequences from eukaryotic or viral genes. For example, they can be promoter sequences derived from the genome of the cell whose infection is desired. Similarly, they can be promoter sequences derived from the genome of a virus, including the adenovirus used. In this respect, promoters which may be mentioned are, for example, the promoters E1A, MLP, CMV, LTR-RSV, etc. In addition, these expression sequences can be modified by the addition of activation sequences, regulatory sequences, or sequences which allow tissue-specific expression. It can in fact be particularly advantageous to use expression signals which are active specifically or mainly in the cells of the cornea, in such a way that the DNA sequence is expressed and produces its effect only when the vector has actually infected these cells.

The nucleic acid encoding one or more anti-angiogenic factors is introduced into a vector. For the purposes of the present invention, “vector” is taken to mean any means enabling the transfer of a nucleic acid into a host cell, preferably into the tissues of the eye and more particularly into the cornea. The term “vector” comprises viral and non-viral vectors for transferring a nucleic acid into a cell in vivo or ex vivo. One type of vector for the implementation of the invention may be for example a plasmid, a cosmid or any DNA not encapsidated by a virus, a phage, an artificial chromosome, a recombinant virus, etc. It is preferably a plasmid or a recombinant virus.

Among the vectors of plasmid type, mention may be made of any cloning plasmid and/or expression plasmid known to persons skilled in the art, and which generally comprises an origin of replication. Mention may also be made of plasmids carrying origins of replication and/or sophisticated selection markers as described, for example, in applications WO 96/26270 and WO 97/10343.

Among the vectors of recombinant virus type, mention may preferably be made of viruses which are adenoviruses, retroviruses, herpesvirus, lentiviruses, recombinant adeno-associated viruses or SV40. The construction of this type of replication-defective recombinant virus has been widely described in the literature, as have the infecting properties of these vectors (see in particular S. Baeck and K. L. March (1998), Circul. Research vol. 82, pp. 295-305), T. Shenk, B. N. Fields, D. M. Knipe, P. M. Howley et al. (1996), Adenoviridae: the viruses and their replication (in virology). pp 211-2148, EDS—Ravenspublishers/Philadelphia, P. Yeh and M. Perricaudet (1997), FASEB Vol. 11, pp. 615-623.

A recombinant virus which is particularly preferred for the implementation of the invention is a defective recombinant adenovirus.

Adenoviruses are viruses with linear double-stranded DNA about 36 kb (kilobases) long. Various serotypes exist thereof, whose structure and properties vary little, but which have a comparable genetic organization. More particularly, recombinant adenoviruses can be of human or animal origin. As regards adenoviruses of human origin, mention may preferably be made of those classified in group C, in particular the adenoviruses of type 2 (Ad2), 5 (Ad5), 7 (Ad7) or 12 (Ad12). Among the various adenoviruses of animal origin, mention may preferably be made of adenoviruses of canine origin and in particular all the strains of the CAV2 adenovirus [Manhattan strain or A26/61 strain (ATCC VR-800) for example]. Other adenoviruses of animal origin are cited in particular in application WO 94/26914 which is incorporated herein by way of reference.

The adenovirus genome comprises in particular an inverted repeat sequence (ITR) at each end, an encapsidation sequence (Psi), early genes and late genes. The principal early genes are contained in the regions E1, E2, E3 and E4. Among these, the genes contained in particular in the E1 region are required for viral propagation. The principal late genes are contained in the regions L1 to L5. The genome of the Ad5 adenovirus has been entirely sequenced and is available on database (see in particular Genbank M73260). Similarly, portions or even the whole of other adenoviral genomes (Ad2, Ad7, Ad12, etc.) have also been sequenced.

Various constructs derived from the adenoviruses, incorporating various therapeutic genes, have been prepared for their use as recombinant vectors. In each of these constructs the adenovirus has been modified in such a way as to make it incapable of replicating in the infected cell. Thus, the constructs described in the prior art are adenoviruses deleted of the E1 region, which is essential for viral replication, into which region the heterologous DNA sequences are inserted (Levrero et al., Gene 101 (1991) 195; Gosh-Choudhury et al., Gene 50 (1986) 161). Moreover, to improve the properties of the vector, it has been proposed to create other deletions or modifications in the genome of the adenovirus. Thus, a heat-sensitive point mutation has been introduced into the mutant ts125, which makes it possible to inactivate the 72 kDa DNA binding protein (DBP) (Van der Vliet et al., J. Virol., 1975, 15(2) 348-354). Other vectors comprise a deletion of another region which is essential to viral replication and/or propagation; the E4 region. The E4 region is in fact involved in the regulation of expression of the late genes, in the stability of the late nuclear RNAs, in the extinction of expression of the host cell's proteins and in the efficacy of the viral DNA replication. Adenoviral vectors in which the E1 and E4 regions are deleted thus have background transcription and very reduced viral gene expression. Such vectors have been described, for example, in applications WO 94/28152, WO 95/02697 and WO 96/22378). In addition, vectors carrying a modification in the IVa2 gene have also been described (WO 96/10088).

In a preferred embodiment of the invention, the recombinant adenovirus is a group C human adenovirus. More preferably, it is an Ad2 or Ad5 adenovirus.

Advantageously, the recombinant adenovirus used in the context of the invention comprises a deletion in the E1 region of its genome. Even more particularly, it comprises a deletion of the E1a and E1b regions. As examples, mention may be made of deletions affecting nucleotides 454-3328; 386-3446 or 357-4020 (with reference to the Ad5 genome).

According to another variant, the recombinant adenovirus used in the context of the invention also comprises a deletion in the E4 region of its genome. More particularly, the deletion in the E4 region affects all the open frames. As specific examples, mention may be made of the deletions 33466-35535 or 33093-35535. Other types of deletion in the E4 region are described in applications WO 95/02697 and WO 96/22378, incorporated herein by way of reference.

The expression cassette containing the nucleic acid encoding an anti-angiogenic factor can be inserted into various sites of the recombinant genome. It can be inserted into the E1, E3 or E4 region as a replacement for, or in addition to, the deleted sequences. It can also be inserted into any other site, other than the sequences required in cis for virus production (ITR sequences and encapsidation sequence).

For the purposes of the present invention “expression cassette” of a nucleic acid is taken to mean a DNA fragment which can be inserted into a vector at specific restriction sites; the DNA fragment comprises, besides the nucleotide sequence encoding an RNA or a polypeptide of interest, the sequences required for the expression (enhancer(s), promoter(s), polyadenylation sequence, etc.) of said sequence of interest. The DNA fragment and the restriction sites are designed so as to ensure insertion of said fragment into an appropriate reading frame for transcription and/or translation.

Recombinant adenoviruses are produced in an encapsidation line, i.e. a line of cells which are capable of transcomplementing one or more of the deficient functions in the recombinant adenoviral genome. Among the encapsidation lines known to persons skilled in the art, mention may be made for example of the 293 line into which has been inserted part of the adenovirus genome. More specifically, the 293 line is a line of human embryonic kidney cells containing the left end (about 11-12%) of the genome of the adenovirus serotype 5 (Ad5), which comprises the left ITR, the encapsidation region, the E1 region, including E1a and E1b, the region encoding the protein pIX and part of the region encoding the protein pIVa2. This line is capable of transcomplementing recombinant adenoviruses which are defective for the E1 region, i.e. devoid of all or part of the E1 region, and of producing viral stocks with high titers. This line is also capable of producing, at permissive temperature (32° C.), stocks of virus comprising, in addition, the heat-sensitive E2 mutation. Other cell lines capable of complementing the E1 region have been described, based in particular on A549 human lung carcinoma cells (WO 94/28152) or on human retinoblasts (Hum. Gen. Ther. (1996) 215). Moreover, lines capable of transcomplementing several functions of the adenovirus have also been described. In particular, mention may be made of lines which complement the E1 and E4 regions (Yeh et al., J. Virol. Vol. 70 (1996) pp. 559-565; Cancer Gen. Ther. 2 (1995) 322; Krougliak et al., Hum. Gen. Ther. 6 (1995) 1575) and lines which complement the E1 and E2 regions (WO 94/28152, WO 95/02697, WO 95/27071).

Recombinant adenoviruses are usually produced by introduction of the viral DNA into the encapsidation line, followed by lysis of the cells after about 2 or 3 days (the kinetics of the adenoviral cycle being 24 to 36 hours). To implement the process, the viral DNA introduced can be the complete recombinant viral genome optionally constructed in a bacterium (WO 96/25506) or in a yeast (WO 95/03400), which is transfected into the cells. It can also be a recombinant virus which is used to infect the encapsidation line. The viral DNA can also be introduced in the form of fragments each carrying part of the recombinant viral genome and a zone of homology which makes it possible, after introduction into the encapsidation cell, to reconstitute the recombinant viral genome by homologous recombination between the various fragments.

After the cell lysis, the recombinant viral particles are isolated by centrifugation in a cesium chloride gradient. An alternative method has been described in application WO 98/00528 incorporated herein by way of reference.

As an example of a suitable vector for implementing the present invention, mention may be made in particular of: the defective recombinant adenovirus comprising the gene encoding the fragment ATF (Ad.CMV.ATF) as described in application WO 98/49321 incorporated herein by way of reference, the defective recombinant adenovirus comprising the gene encoding angiostatin K3 (Ad-K3) as described in application WO 98/49321.

The invention also relates to a pharmaceutical composition comprising a vector as described above and a physiologically acceptable vehicle for a formulation intended to be administered into the eye, in particular by instillation. It can be in particular isotonic, sterile, saline solutions (monosodium phosphate, disodium phosphate, sodium chloride, potassium chloride, calcium chloride or magnesium chloride, etc., or mixtures of such salts) or dry compositions, in particular lyophilized compositions, which by addition of sterilized water or physiological saline, depending on the case in question, allow the making-up of solutes intended for ocular instillation.

The doses used for the instillation or the impregnation of the contact lenses can be adapted as a function of various parameters and in particular as a function of the method of administration used, the chemical nature of the contact lenses, the gene to be expressed, or alternatively of the duration of the desired expression. Generally, the recombinant viruses according to the invention are formulated and administered in the form of doses between 10⁴ and 10¹⁴ pfu, and preferably 10⁶ to 10¹⁰ pfu. The term pfu (“plaque forming unit”) corresponds to the infectious power of a viral solution, and is determined by infecting an appropriate cell culture and measuring the number of plaques of infected cells. The techniques for determining the pfu titer of a viral solution are well documented in the literature.

In addition, the compositions according to the invention can also comprise a chemical or biochemical transfer agent. The term “chemical or biochemical transfer agent” is taken to mean any compound (i.e. other than a recombinant virus) which facilitates the penetration of a nucleic acid into a cell. It can be cationic non-viral agents such as cationic lipids, peptides, polymers (Polyethylene Imine, Polylysine), nanoparticles; or non-cationic non-viral agents such as non-cationic liposomes, polymers or non-cationic nanoparticles.

According to a preferred embodiment, the compositions according to the invention comprise a defective recombinant vector which comprises a gene encoding an anti-angiogenic factor, and are formulated for intraocular instillation. Advantageously, the compositions of the invention comprise from 10⁴ to 10¹⁴ pfu, and preferably from 10⁶ to 10¹⁰ pfu. The titer of the solutions used for the impregnation of the contact lenses is between 1×10⁶ and 1×10¹² pfu/ml and preferably between 1×10⁸ and 1×10¹⁰ pfu/ml.

The invention also relates to a kit comprising a recipient which contains a composition as previously mentioned and at least one contact lens, and the use of the kit for preparing a medicinal product intended to be administered via impregnation of a lens, for the prevention, improvement and/or treatment of corneal neovascularization.

The contact lenses which can be used in the context of the present invention are well known to persons skilled in the art and are preferably soft contact lenses, as described in particular in Künzler et al. (Chemistry & Industry, 651-655 (1995); Künzler et al. (TRIP, Vol. 4 (2) 52-59 (1996); J. Singh et al. (J.M.S. Rev. Macromol. Chem. Phys. C32(3&4), 521-534 (1992)); J. C. Wheeler et al. (Journal of Long-Term Effects of Medical Implants, 6 (3&4): 207-217 (1996)).

A subject of the invention is also a process for preparing a medicinal product which is useful for the prevention, improvement and/or treatment of corneal neovascularization, characterized in that a recombinant vector comprising a nucleic acid encoding an anti-angiogenic vector is mixed with one or more compatible and pharmaceutically acceptable adjuvants.

The invention also relates to a method for treating a mammal, and in particular man, displaying a corneal neovascularization, which comprises the administration of an effective amount of a recombinant vector comprising a nucleic acid encoding an anti-angiogenic vector, via impregnation of a contact lens.

The present invention will be described in greater detail with the aid of the following examples, which should be considered as illustrative and non-limiting.

### LEGEND TO FIGURES

FIG. 1: Detection of β-galactosidase expression by immunohistochemistry on paraffin-embedded sections (5 μm) of adult rat cornea which has been in contact with a lens impregnated with Ad. β-gal 2×10⁷ pfu), for 72 h. Hemalun staining. (A) Cornea which has neovascularized following superficial scraping, magnification×265. (B) nuclear labeling of epithelial cells revealed with an antiβ-gal antibody, magnification×1310. (C) Avascular normal control cornea, magnification×525. (e: epithelium; en: endothelium; neovx: neovessels; s: stroma).

FIG. 2: Photograph of a corneal neovascularization (black triangle) induced by sutures (black arrows) placed in the cornea of an adult rat two weeks earlier. Magnification×24.

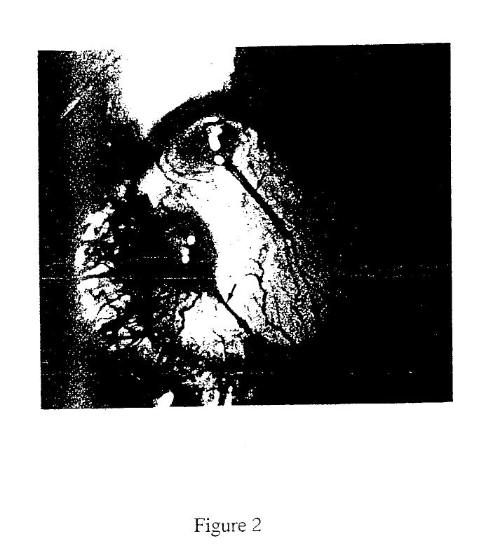

FIG. 3: Preventive anti-angiogenic effect of a lens impregnated with an adenoviral solution (Ad.CMV.ATF, Figure (B) and (D); AdK3, Figure (A) and (C)). Magnification×20. (A) and (B) correspond to corneas which have remained in contact with the lens for one week. (C) and (D) correspond to corneas which have remained in contact with the lens for two weeks.

### MATERIALS AND METHODS

General Molecular Biology Techniques

The methods conventionally used in molecular biology, such as preparative extractions of plasmid DNA, centrifugation of plasmid DNA in a cesium chloride gradient, agarose or acrylamide gel electrophoresis, purification of DNA fragments by electroelution, extraction of proteins with phenol or phenol/chloroform, ethanol or isopropanol precipitation of DNA in saline medium, transformation in Escherichia coli, etc., are well known to persons skilled in the art and are widely described in the literature [Maniatis T. et al., “Molecular Cloning, a Laboratory Manual”, Cold Spring Harbor Laboratory, Cold Spring Harbor, N.Y., 1982; Ausubel F. M. et al. (eds), “Current Protocols in Molecular Biology”, John Wiley & Sons, New York, 1987.

For ligations, the DNA fragments can be separated according to their size by agarose or acrylamide gel electrophoresis, extracted with phenol or with a phenol/chloroform mixture, precipitated with ethanol and then incubated in the presence of T4 phage DNA ligase (Biolabs) according to the supplier's recommendations.

Filling of the protruding 5′ ends can be carried out by the Klenow fragment of E. coli DNA polymerase I (Biolabs) according to the supplier's specifications. Destruction of the protruding 3′ ends is carried out in the presence of T4 phage DNA polymerase (Biolabs) used according to the manufacturer's recommendations. Destruction of the protruding 5′ ends is carried out by controlled treatment with S1 nuclease.

Site-directed mutagenesis in vitro by synthetic oligodeoxynucleotides can be carried out according to the method developed by Taylor et al. [Nucleic Acids Res. 13 (1985) 8749-8764] using the kit distributed by Amersham.

The enzymatic amplification of DNA fragments by the so-called PCR technique [Polymerase-catalyzed Chain Reaction, Saiki R. K. et al., Science 230 (1985) 1350-1354; Mullis K. B. and Faloona F. A., Meth. Enzym. 155 (1987) 335-350] can be carried out using a DNA thermal cycler (Perkin Elmer Cetus) according to the manufacturer's specifications.

Verification of the nucleotide sequences can be carried out by the method developed by Sanger et al. [Proc. Natl. Acad. Sci. USA, 74 (1977) 5463-5467] using the kit distributed by Amersham.

Model of Corneal Neovascularization in Rats

Thirty 150 g adult male Wistar rats are anesthetized by an intraperitoneal injection of 15 mg of Ketamine and 1.5 mg of Xylazine. The general anesthetic is supplemented with topical anesthesia with the aid of a drop of oxybuprocaine hydrochloride (Novesine®) onto the cornea. Two models were: a model by interrupted sutures and a model by scraping.

Model by interrupted sutures: twenty rats were used to evaluate this method of induction of corneal neovascularization. Three interrupted sutures (Nylon 8-0, 50 μm needle) are positioned across the central cornea of the right eye of the animal under aseptic conditions, using an operating microscope (Zeiss). The unoperated left eye serves as a control. The corneas are monitored with a photographic biomicroscope, for the development of corneal neovessels. To quantify the corneal neovascularization, under double blind conditions, the eye is divided into four identical corneal quadrants and the neovascularization of each is graduated from 0 to 3, by divisions of 0.5. Thus, the score for each eye stretches from 0 to 12 (W. Streilen et al. 1996, Invest. Ophthamol. Vis. Sci. 37: 413-424).

Model by scraping: the corneal and limbic epithelia of the right eye of ten rats are removed by surgical scraping for 10 min. With the aid of a 15° scalpel, the by excising with 100° ethanol (EtOH) under an operating microscope (Zeiss), (A. HUANG et al. 1988, Ophthalmology 95, 228-235). The bare corneas heal in about ten days and vascularize. The corneal neovascularization is then evaluated as described in the model by interrupted sutures, the unoperated left eye serving as a control.

Preparing and putting in the lenses: disposable soft lenses are cut with a hole punch of diameter smaller than that of the rat cornea (3 mm) and are then put with adenoviral solutions AdK3, Ad.CMV.ATF or Ad.CMVβgal, respectively of 4.5×10⁵ pfu (plaque forming unit), 10⁶ pfu and 2×10⁷ pfu for each lens, for one to two hours at room temperature. Control lenses are also prepared with 10% glycerol, the vehicle in which each adenovirus is diluted. Once the lens has been put in on the right cornea of the rat, the eye is carefully redosed by carrying out a tarsorraphy (silk 6-0, 50 μm needle), the unoperated left eye serving as a control.

Expression of the transgene by the adenoviruses is detected using the techniques (1) of revelation by colorimetry on frozen sections, for revealing the β-galactosidase only, or (2) of immunohistochemistry on paraffin sections.

(1) Newborn Wistar rats receive an Ad.CMVβgal lens on the right eye. For this, the animals are anesthetized by immersion in ice and then the eyelid is carefully cut parallel to its future line of opening using Dowell sissors angled at 45° and microsurgical forceps. Once the lens has been positioned on the cornea, the eye is reclosed with the aid of silk 6.0.

(2) Twelve adult rats without corneal neovascularization (four animals per batch) receive either an AdK3 lens, an Ad.CMV.ATF lens or an Ad.CMVβgal lens on the right eye, and 3 rats with neovascularization are operated with an Ad.CMVβgal lens. A tarsorraphy is carried out to reclose the operated eye.

Revelation techniques: three different techniques are used to detect the β-galactosidase. The animals are sacrificed by cervical elongation 72 h after putting in the lens, the eyelids are opened and the eyes enucleated with forceps. The β-galactosidase is detected on whole-mounted eye or on 14 μm frozen sections or on 5 μm-thick paraffin sections.

(i) On whole-mounted eye: after fixing in 0.5% glutaraldehyde for 1 h at room temperature, each eye (adult or newborn rat) is immersed in a solution of 1×PBS containing 2 mM of MgCl₂ and then cleared of the residual muscle tissues under a binocular lens (Leica). Each eye is then incubated in a revelation solution of 5 mM K₃Fe(CN)₆, 5 mM K₄Fe(CN)₆, 2 mM MgCl₂, 1 mg/ml 5-bromo-4-chloro-3-indolyl-b-D galactoside (X-gal) for 2 h at 37° C. After several washes in 1×PBS, the eye is photographed under a binocular lens.

(ii) 14 μm frozen sections: the newborn rat eyes are immediately fixed in 0.5% glutaraldehyde for 1 h at room temperature and are then cryoprotected in 15% sucrose for 30 min. The eyes are then placed in cupules of solid plastic containing an embedding medium (Tissue Tek) which, once frozen in isopentane at −30° C., makes it possible to conserve the tissues at −80° C. The eyes are cut at 14 μm with a cryostat (Leica). The revelation solution is then deposited directly onto the sections, in a humid chamber, for 2 h at 37° C. After three rinses of 10 min. in 1×PBS, the sections are counterstained with 1% neutral red, dehydrated in baths of increasing degrees of alcohol: 80°, 95° and 100° and then mounted between slide and coverslip in Eukitt.

(iii) 5 μm-thick paraffin sections: the method for detecting β-galactosidase by immunohistochemistry is a technique which is much more sensitive than the conventional method of revelation by colorimetry. After 48 hours of immersion in Davidson's fixative, the adult rat eyes are careful dissected to remove the lens, placed in a plastic cassette and put into the basket of an automatic machine which performs the following treatment cycle: 10% Formol, 30 min.; 700 EtOH, 1 h; 80° EtOH, 1 h; 100° EtOH, 5 times 1 h; xylene, 2 times 1 h 30; paraffin, 4 times 1 h. The globes are then embedded in paraffin and cut sagittally, parallel to the optic nerve, in 5 μm sections using a microtome (Leica). The sections are collected on pretreated slides (DAKO).

Fixing the sections: to benefit from an immunohistochemical revelation, with an anti-β-galactosidase anbitody (TEBU), the slides are dried in an incubator at 56° C. for 48 h then deparaffinized in 2 xylene baths of 15 min. and 2 100° EtOH baths of 10 min. They are then rehydrated for a few minutes in running water.

Blocking and permeabilization: the slides are placed in a 0.01 M citrate buffer; pH 6, first at room temperature for 5 min., then in a microwave oven at power 8 (750 watts), 2 times 5 min. Having returned to room temperature, the sections are rinsed in osmosed water and circled with a DAKO pen. In order to eliminate the tissue peroxidases which can interfere with the revelation complex and thus make the reaction uninterpretable, we use 3% H₂O₂ for 10 min. The slides are then preincubated for 30 min. in a 1×PBS, 2 g/l gelatin, 0.25% Triton and 3% bovine serum albumin (BSA) mixture to saturate the aspecific sites.

Labeling: the anti-β-galactosidase rabbit primary antibody is diluted to 1/1000 in a mixture of 1×PBS, 2 g/l gelatin and 0.025% Triton. After 1 h of incubation at room temperature, a brief rinse then four others of 5 min. are carried out with the mixture which was used to dilute the antibody. The anti-rabbit biotinylated secondary antibody, produced in donkeys (DAKO), is diluted to 1/200 in 1×PBS supplemented with gelatin at 2 g/l and applied for 30 min. The slides are then rinsed, first rapidly then 4 times 5 min., with the dilution medium before adding the streptavidin/peroxidase (DAKO) revelation complex diluted at 1/400 in the same mixture, for 30 min. After three 5 min. washes, the color-forming substrate, diaminobenzidine (DAB), is added. The revelation time varies from 2 to 20 min. The reaction is stopped by plunging the slides into osmosed water. They are then counterstained in Hemalun for 1 min., rinsed rapidly in osmosed water then differentiated in LiCO₃ for a few seconds. Finally, they are dehydrated then conserved in xylene until mounting between slide and coverslip in Eukitt.

### EXAMPLE 1

### Construction of the Defective Recombinant Adenoviruses Ad.CMV.ATF, AdK3 and Ad.CMVβgal

The vectors were constructed according to the general method described by Crouzet et al (PNAS vol. 94 p. 1414, 1997). The details of the construction of the vectors Ad.CMV.ATF and AdK3 have been described in application WO 98/49321 incorporated herein by way of reference.

For the AdK3, the 1 Kb transgene corresponds to the N-terminal fragment of human plasminogen (upto residue 333) and includes the first 3 kringle domains of the angiostatin molecule.

For the Ad.CMV.ATF, the 0.5 Kb transgene is a cDNA encoding the N-terminal fragment of murine uPA (amino acids 1 to 135) and the transgene of the Ad.CMVβgal comprises a 3.1 Kb cDNA encoding the Escherichia coli reporter gene lacZ.

The production and secretion of adenoviral molecules from the constructs were verified, respectively, by Northern blotting and Western blotting. In vivo tests of inhibition of tumor growth, angiogenesis and tumorigenesis, as well as in vitro tests of inhibition of bFGF-stimulated cellular proliferation, were carried out for the AdK3 and Ad.CMV.ATF. The X-gal activity of the Ad.CMVβgal was also controlled.

The average titer of the stock solutions of the recombinant adenoviruses used is 4.5×10⁸ pfu/ml for the AdK3, 10⁹ pfu/ml for the Ad.CMV.ATF and 6.9×10¹⁰ pfu/ml for the Ad.CMVβgal.

### EXAMPLE 2

### Effect of a Vector Encoding an Anti-Angiogenic Factor in a Model of Corneal Neovascularization in Rats

The effect of the lens on the right eye of the animal is studied as a function (1) of the duration of the contact with the cornea—one or two weeks—(2) of the presence or absence of the vector encoding an anti-angiogenic factor—(3) of the neovascularization or otherwise of the cornea.

To investigate a “preventive” therapeutic effect of the adenovirus on the corneal neovascularization, the lens is positioned on the cornea immediately after putting in 3 interrupted sutures, whereas a “curative” effect is studied by putting the lens on to eyes which have already been showing a corneal neovascularization for one or two weeks.

Each adenovirus is tested on a batch of 10 rats: 5 to evaluate the preventive effect and 5 for the curative effect. A batch of 10 rats with a lens impregnated with a vehicle serves as a control.

Evaluation of the Two Animal Models of Corneal Neovascularization

The model of induction of corneal neovessels by putting in three interrupted sutures is very efficient since 100% of the operated animals show a corneal neovascularization and the cornea of the contralateral control eyes is avascular. The vessels are attracted towards the center of the cornea from the limbic vascular arches and proliferate in the superficial layers of the cornea from the first week following the intervention.

The average score of each eye, which indicates the development of the corneal neovascularization, is 7±2 two weeks after putting in the sutures. At that time the corneal neovascularization is maximal. In the model of induction by superficial scraping of the cornea only 70% of the rats develop a corneal neovascularization. However, it is more homogeneous than that of the previous model because all the four quadrants of the eye participate in the development of the neovascularization, unlike that which is observed in the sutures model where the number of quadrants involved is variable. This variability of the area of the neovascularized corneal surface is strictly dependent on the position of the sutures relative to the limbus, on their depth in the stroma and on their size. The average score for each eye is 8±2 two weeks after scraping.

On a paraffin-embedded section of cornea, which has been stained with a mixture of periodic acid/Schiff's base/hematoxylin, the neovessels appear predominantly in the corneal epithelium, but also in the stroma and in contact with the corneal endothelium.

Efficacy of the Transfer of the Adenoviral Vectors by Prior Impregnation of a Contact Lens

Macroscopic results: after incubation with the X-gal solution, the ocular cupules of all the eyes having been exposed to a lens impregnated with the β-galactosidase adenovirus at 2×10⁷ pfu/μl show a blue staining which is punctate and very widespread over the entire cornea of the newborn rat and over a large region of the cornea of the adult animals. The blue staining is found on none of the control eyes.

Microscopic results: the labeling obtained by colorimetric revelation of β-galactosidase on frozen sections of normal corneas is predominant in the nucleus of the corneal epithelial cells.

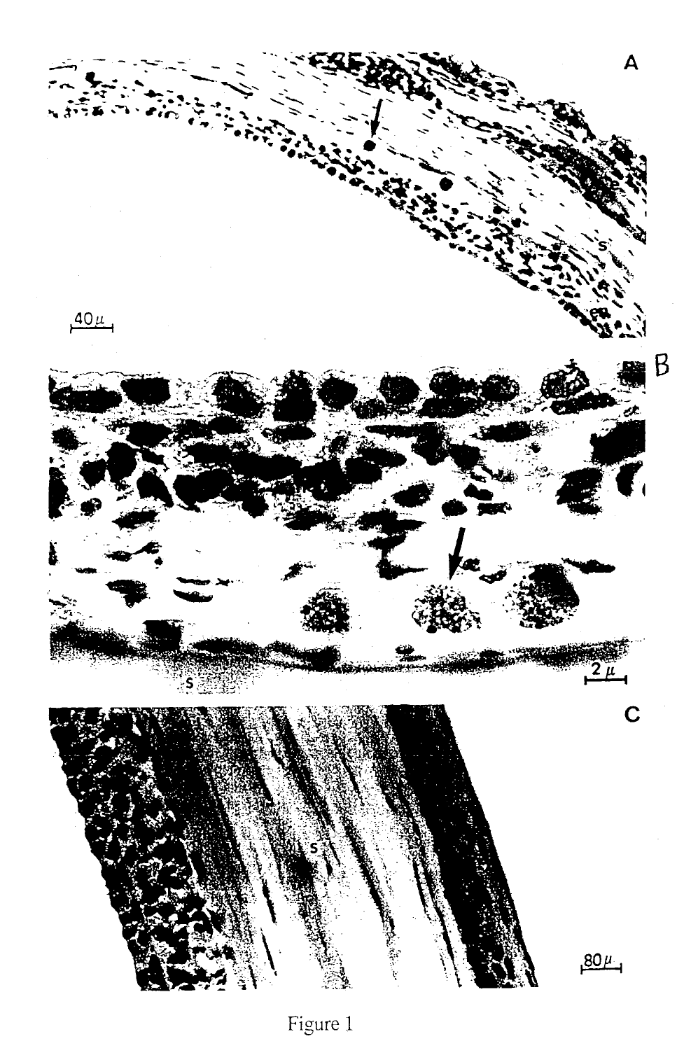

In paraffin-embedded sections of neovascularized corneas the labeling obtained by immunohistochemistry is found principally in the nuclei of the corneal endothelial cells, but also in that of the corneal epithelial cells and ciliary body epithelial cells. The keratocytes of the corneal stroma and of the pathological intrastromal endothelial cells are also labeled (FIG. 1). Despite the nuclear localization signal which is added to the lacZ transgene, certain cells show labeling in the cytoplasm, whatever the revelation technique.

Identical results are obtained with the aid of adenovirus encoding the murine ATF or human angiostatin. This was demonstrated by specific immunohistochemical reactions performed on paraffin-embedded sections of rat eyeballs.

Anti-Angiogenic Effect of the Lenses Preimpregnated with Adenovirus Ad.CMV.ATF and AdK3

Lenses impregnated with vehicle and positioned on a cornea without neovascularization have no significant angiogenic effect.

No inflammatory phenomenon and no corneal neovascularization are induced by the lenses soaked in Ad.CMVβgal at 2×10⁷ pfu/μl.

Similarly, the Ad.CMV.ATF (10⁶ pfu/μl) lenses and AdK3 (4.5×10⁵ pfu/μl) lenses induce no inflammatory reaction on the cornea, no neovascularization, nor any disappearance of the limbic vascularization.

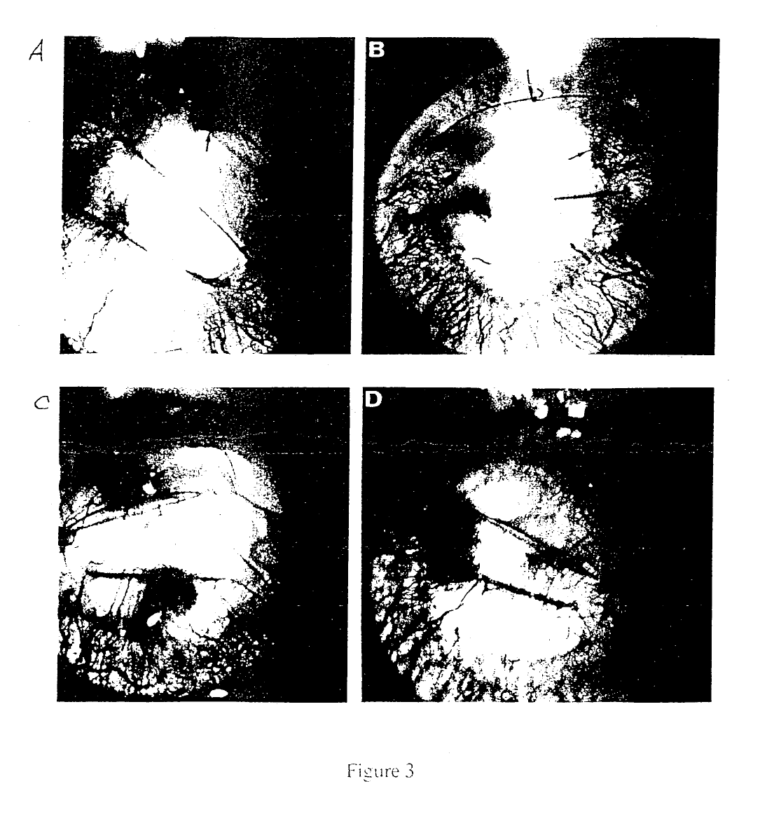

An Ad.CMV.ATF-soaked lens positioned on a cornea immediately after putting in the three interrupted sutures prevents the development of the neovascularization which is expected subsequent to putting in these three sutures, during the first week. This effect is still occurring after two weeks. The same anti-angiogenic effect is observed with an AdK3-soaked lens at the end of one week of contact with the cornea (FIG. 3).

1. The use of a vector comprising a nucleic acid encoding an anti-angiogenic factor for the preparation of a pharmaceutical composition intended to be administered by impregnation of a soft lens and application of said lens on to the cornea, for the prevention, improvement and/or treatment of corneal neovascularization.

2. The use as claimed in claim 1, characterized in that the nucleic acid encoding the anti-angiogenic is a nucleic acid encoding a polypeptide chosen from the N-terminal fragment of the plasminogen activator uPA (ATF), angiostatin, endostatin, the 16 kDa fragment of prolactin or platelet factor 4 (PF-4) or a combination of nucleic acids encoding at least two of these factors.

3. The use as claimed in claim 2, characterized in that the anti-angiogenic factor is chosen from the N-terminal fragment of the plasminogen activator uPA (ATF) and angiostatin K3.

4. The use as claimed in one of claims 1 to 3, characterized in that the vector is a plasmid, a cosmid or any DNA not encapsidated by a virus.

5. The use as claimed in one of claims 1 to 3, characterized in that the vector is a recombinant virus, preferably derived from an adenovirus, a retrovirus, a lentivirus, a herpesvirus or an adeno-associated virus.

6. The use as claimed in claim 5, characterized in that the recombinant virus is a defective recombinant adenovirus.

7. The use as claimed in claim 5 or 6 for preparing a pharmaceutical composition intended for administration by intraocular instillation, comprising from 1×10⁶ and 1×10¹² pfu/ml and preferably between 1×10⁸ and 1×10¹⁰ pfu/ml.

8. A process for preparing a medicinal product which is useful for the prevention, improvement and/or treatment of corneal neovascularization, characterized in that a recombinant vector comprising a nucleic acid encoding an anti-angiogenic factor is mixed with one or more compatible and pharmaceutically acceptable adjuvants.

9. A pharmaceutical composition comprising a defective recombinant vector which comprises at least one nucleic acid encoding an anti-angiogenic factor, characterized in that it is formulated for intraocular administration.

10. The pharmaceutical composition as claimed in claim 9, characterized in that it comprises from 1×10⁶ and 1×10¹² pfu/ml and preferably between 1×10⁸ and 1×10¹⁰ pfu/ml.

11. A kit comprising a recipient which contains a composition as claimed in either of claims 9 and 10 and at least one contact lens.

12. The use of the kit as claimed in claim 11 for the preparation of a medicinal product intended to be administered via impregnation of a lens, for the prevention, improvement and/or treatment of corneal neovascularization.
